SEQUENCE LISTING (4 sequences)

SEQ ID NO: 1 - DNA, 21 bp, Artificial
  Synthetic oligonucleotide primer for miox gene (forward)
cccatggcgatctctgttgag

SEQ ID NO: 2 - DNA, 21 bp, Artificial
  Synthetic oligonucleotide primer for miox gene (reverse)
ccggatcctcaccacctcaag

SEQ ID NO: 3 - DNA, 954 bp, Arabidopsis thaliana
  Source (1)..(954): nucleic acid sequence of A. thaliana miox4 cDNA PCR product
atgacgatctctgttgagaagccgatttttgaagaagaggtttctgcattcgagaagagtggggacaatatcggagagttgaaattggacggaggattttcgatgccgaaaatggacaccaatgacgacgaagcttttttggctcctgagatgaatgcatttggccgccaattcagggactacgatgttgagagtgagaggcaaaagggcgtcgaagagttttacagattacgacacatcaaccaaactgtcgactttgtgaaaaagatgagggctgaatatggaaaattagataaaatggtgatgagcatttgggaatgttgtgagcttctcaatgaggttgtggatgagagtgatccagatcttgacgagccccagattcagcatttgcttcaatctgccgaagccatccgcaaagattaccctaatgaagattggcttcatctgaccgctcttatccatgatcttgggaaagttattactcttccacaattcggaggacttcctcaatgggctgttgttggtgacacattccctgttggatgtgcatttgatgaatctaacgtacatcacaagtactttgtggaaaacccagattttcacaacgaaacctacaacactaaaaatgggatttactctgaagggtgtggattaaacaatgtcatgatgtcttggggccatgacgactacatgtacctggtggctaaagaaaacggaagtaccttgccgtcggctggacagtttatcataagataccactccttttaccctttgcacacggctggagaatacacccatcttatgaacgaggaagacaaggagaatctgaagtggctacacgttttcaacaagtacgacttgtacagtaagagcaaagttcacgttgatgtggagaaggtcgagccttactacatgtctcttatcaagaaatatttcccggaaaacttgaggtgg

SEQ ID NO: 4 - protein, 318 aa, Arabidopsis thaliana
  Source (1)..(318): Amino acid sequence of A. thaliana miox4 cDNA product
MTISVEKPIFEEEVSAFEKSGDNIGELKLDGGFSMPKMDTNDDEAFLAPEMNAFGRQFRDYDVESERQKGVEEFYRLRHINQTVDFVKKMRAEYGKLDKMVMSIWECCELLNEVVDESDPDLDEPQIQHLLQSAEAIRKDYPNEDWLHLTALIHDLGKVITLPQFGGLPQWAVVGDTFPVGCAFDESNVHHKYFVENPDFHNETYNTKNGIYSEGCGLNNVMMSWGHDDYMYLVAKENGSTLPSAGQFIIRYHSFYPLHTAGEYTHLMNEEDKENLKWLHVFNKYDLYSKSKVHVDVEKVEPYYMSLIKKYFPENLRW

(19) United States
(12) Patent Application Publication
(10) Pub. No.: US 2006/0212960 A1
(43) Pub. Date: Sep. 21, 2006

(54) Manipulation of ascorbic acid levels in plants

(76) Inventors: Craig L. Nessler, Blacksburg, VA (US); Argelia Lorence, Blacksburg, VA (US); Boris I. Chevone, Blacksburg, VA (US); Pedro P.J. Mendes, Blacksburg, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 10/538,885

(22) PCT Filed: Sep. 8, 2003
(86) PCT No.: PCT/US03/27779

§ 371 Date: Mar. 29, 2006

Related U.S. Application Data
(60) Provisional application No. 60/433,787, filed on Dec. 17, 2002.

(51) Int. Cl. A01H 1/00; C12N 15/82; A01H 5/00

(52) U.S. Cl. 800/278; 800/317.3

(57) ABSTRACT

Methods for increasing the vitamin C content of plants are provided by transforming the plants with genes encoding a novel vitamin C biosynthetic pathway. Vitamin C production is increased in the resulting transgenic plants, providing, for example, higher nutritional value and longer shelf-life of produce. Further, the leaves of air-cured varieties of tobacco transformed in this manner contain lower levels of highly carcinogenic tobacco specific nitrosamines (TSNAs).

This invention was made using funds from grants numbered 427128 NSF and 428972 USDA. The United States government may have certain rights in this invention.

### DESCRIPTION

### Field of the Invention

The invention generally relates to methods for increasing the vitamin C (L-ascorbic acid, AsA) content of plants. In particular, the invention provides genes encoding a novel AsA biosynthetic pathway and transgenic plants transformed with those genes. The transgenic plants produce higher levels of AsA than corresponding non-transformed plants. When the transgenic plant is an air-cured variety of tobacco, the cured leaves of the tobacco plant contain lower levels of tobacco-specific nitrosamines.

### BACKGROUND OF THE INVENTION

L-ascorbic acid (AsA, vitamin C) is the predominant antioxidant in plant cells and has important antioxidant and metabolic functions in both plants and animals. AsA may reach a concentration of 1-5 mM in the leaves of some plants and over 20 mM in chloroplasts. Since AsA was first isolated, there have been numerous reports on its role regulating redox potential during photosynthesis, environment-induced oxidative stress (ozone, UV, high light, SO₂, etc), and during wound- and pathogen-induced oxidative processes. In both plants and animals, AsA is also important as a cofactor for a large number of key enzymes (Loewus and Loewus, 1987; Smirnmoff et al, 2001; Arrigoni and De Tullio, 2002). There is emerging evidence that AsA is involved in photoprotection, metal and xenobiotic detoxification, the cell cycle, cell wall growth, and cell expansion (Franceschi and Tarlyn, 2002; Smirnoff, 2000; Smirnoff and Wheeler, 2000). Interestingly, a recent study indicates that leaf AsA content can also modulate the expression of genes involved in plant defense as well as regulate genes that control development through hormone signaling (Pastori et al., 2003).

The antioxidant property of AsA is one of its major functions in humans. However, all primates, including humans, have lost the ability to synthesize AsA. Because AsA can neither be produced nor stored in the primate body, the vitamin must be acquired regularly from dietary sources. Failure to consume adequate levels of AsA is known to result in the development of serious pathological conditions such as scurvy. The primary source of AsA is from plants. However, AsA levels in plant sources vary widely so that insuring adequate consumption is not always straightforward, especially in areas of the world where food sources are inadequate or unpredictable. Thus, it would be desirable to have the ability to increase the level of AsA in fruits and vegetables in order to facilitate the consumption of adequate amounts of the vitamin. In addition, increased levels of AsA would benefit the plants themselves by increasing their ability to withstand oxidative stress during the growing season, thereby decreasing crop damage, increasing yield and perhaps hastening harvest times. Further, increasing the AsA content of plants would help to maintain their condition after harvest, for example, the shelf life of produce would be increased.

Although fresh fruits and vegetables are the major source of AsA in the human diet only limited information is available concerning its route(s) of synthesis in plants. Thus, there is an ongoing need to understand AsA synthesis routes in plants, and to apply that knowledge to discover methodologies which can be used to increase the AsA content of plants.

Other motivations for acquiring the ability to increase the AsA content of plants also exist, particularly with respect to tobacco plants. During the curing of both flue-cured and air-cured tobacco plants, compounds known as tobacco specific nitrosamines (TSNAs) are produced. TSNAs are highly carcinogenic compounds formed from the nitrosation of tobacco alkaloids. In the case of flue-cured tobacco, evidence suggests that TSNAs are formed from the direct interactions of combustion products with the leaf alkaloids. A $57 million program has recently been completed by the major U.S. tobacco companies to convert domestic curing barns so that combustion gases can be vented from barns instead of mixing with the drying leaves. While this conversion appears to have eliminated TSNAs from flue-cured tobacco, no similar simple engineering fix is currently available for air-cured tobacco varieties. In the case of air-cured tobacco, TSNA accumulation appears to result from microbial activity during the prolonged curing process in open air. Reported experiments (Rundlof et al., 2000) indicate that infiltration of air-cured tobacco leaves with ascorbic acid substantially reduces the accumulation of TSNAs. However, such a procedure involves an additional time-consuming step in the processing of tobacco leaves. Thus, there is an ongoing need to provide more convenient, alternative means to introduce vitamin C into tobacco plants in order to lessen the level of TSNAs produced in leaves during air-curing.

### SUMMARY OF THE INVENTION

The present invention provides a novel vitamin C biosynthetic pathway in plants and methods for increasing the AsA content of plants by transformation with one or more genes from the pathway. The resulting transgenic plants have increased nutritional value. Further, the higher AsA content provides other advantages such as a longer shelf life for produce.

In a particular application of the invention, it has been discovered that the leaves of air-cured varieties of tobacco plants transformed in this manner produce increased AsA and contain lower levels of highly carcinogenic tobacco-specific nitrosamines (TSNAs) after curing. Thus, tobacco products made from this type of transgenic tobacco are preferable to those varieties from varieties not transformed in this manner.

Thus, it is an object of this invention to provide a plant that is genetically modified to include at least one gene encoding an enzyme from a AsA biosynthetic pathway, wherein the pathway includes a myo-inositol oxygenase enzyme. The plant may include more than one copy of the gene, and may further include a means to enhance transcription of the gene or genes.

The plant may be for example, lettuce, tobacco, or Arabidopsis. In one embodiment of the invention, the plant is a tobacco plant. In yet another embodiment, the gene which is utilized encodes a myo-inositol oxygenase enzyme.

The present invention further provides a method of increasing an endogenous level of AsA in a plant. The method comprises the step of genetically modifying the plant to contain at least one gene encoding an enzyme from a AsA biosynthetic pathway, wherein said pathway includes a myo-inositol oxygenase enzyme, and wherein the step of genetically modifying the plant results in increasing the intrinsic level of AsA in the plant. The plant may contain more than one copy of the gene, and may further include a means to enhance transcription of the gene or genes. The plant may be, for example, lettuce, tobacco, or Arabidopsis. In one embodiment of the invention, the plant is a tobacco plant. In yet another embodiment, the gene which is utilized encodes a myo-inositol oxygenase enzyme.

The present invention also provides a method for reducing TSNAs in air cured tobacco The method comprises the step of genetically engineering the tobacco to include at least one gene in a AsA biosynthetic pathway, wherein the step of genetically engineering the tobacco results in reduced levels of TSNAs in the tobacco. The tobacco may include more than one copy of the gene, and may further include a means to enhance transcription of the gene or genes. The pathway may include a myo-inositol oxygenase enzyme or a L-gulono-gamma-lactone oxidase enzyme. The gene may be a rodent L-gulono-gamma-lactone oxidase enzyme or a myo-inositol oxygenase enzyme. The method may result in an increase in an endogenous level of AsA in the tobacco.

The present invention also provides tobacco plants that produce elevated levels of vitamin C. The tobacco plants may be produced by genetic engineering or by selective breeding.

### BRIEF DESCRIPTION OF THE DRAWINGS

FIG. 1. Proposed biosynthetic pathways of L-ascorbic acid in plants (reactions 1-12), and animals (reactions 13-20). Enzymes catalyzing the numbered reactions are: 1, methylesterase; 2, D-galacturonate reductase; 3, aldono-lactonase; 4, L-galactono-1,4-lactone dehydrogenase; 5, glucose-6-phosphate isomerase; 6, mannose-6-phosphate isomerase; 7, phosphomannomutase; 8, GDP-mannose pyrophosphorylase; 9, GDP-mannose-3,5-epimerase; 10, phosphodiesterase; 11, sugar phosphatase; 12, L-galactose-1-dehydrogenase; 13, phosphoglucomutase; 14, UDP-glucose pyrophosphorylase; 15, UDP-glucose dehydrogenase; 16, glucuronate-1-phosphate uridylyltransferase; 17, glucurono kinase; 18, glucuronate reductase; 19, aldono lactonase, and 20, guluno-1,4-lactone dehydrogenase. The reaction catalyzed by myo-Inositol (MD oxygenase (MIOX), and the possible pathway from MI to AsA (dashed arrows), are also shown.

FIG. 2. The miox4 message is present in flowers and leaves, tissues with high demand for AsA. (A) RNA expression of miox4 in leaf and flower tissues of 6 week-old A. thaliana wild type plants. (B) Loading control: membrane shown in (A) hybridized with a β-ATPase probe. (C) Ascorbic acid content of the corresponding plant tissues. (n=3)

FIG. 3. Constitutive expression of miox4 in A. thaliana increases the ascorbic acid content of the leaves. (A) RNA expression of miox4 in control (labeled as C, pCAMBIA 1380), and three homozygous transgenic lines, numbered L1, L2, and L3. (B) rRNA of the samples in (A) stained with ethidium bromide is shown as a loading control. (C) Ascorbic acid content in the leaf extract of the corresponding control (C) and transgenic lines L1, L2 and L3. (n=3)

FIG. 4. The nucleic acid sequence of A. thaliana miox4 cDNA PCR product (SEQ ID NO:3).

FIG. 5. The amino acid sequence of A. thaliana miox4 cDNA product (SEQ ID NO:4).

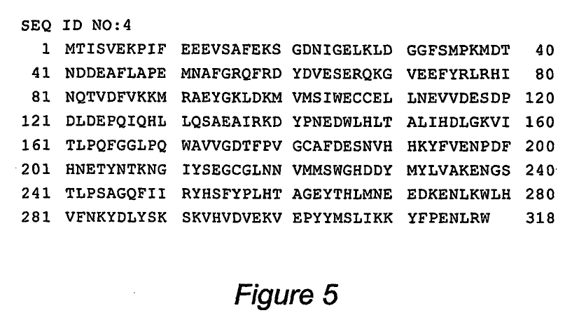

### DETAILED DESCRIPTION OF THE PREFERRED EMBODIMENTS OF THE INVENTION

The present invention identifies a previously unknown pathway for the biosynthesis of AsA in plants. The pathway includes the enzyme myo-inositol oxygenase.

The present invention seeks to 1) enhance AsA production in plants by promoting the pathway (i.e. providing the chemical constituents used in the pathway); and to 2) genetically transform plants to include one or more genes in the pathway.

The invention further provides transgenic plants which have been genetically modified to contain one or more genes encoding one or more enzymes from the novel AsA biosynthetic pathway. In a preferred embodiment, a gene which is used to genetically modify the transgenic plant encodes the enzyme myo-inositol oxygenase (MIOX).

By “transgenic plants that have been genetically modified” we mean that the plants have been genetically engineered to contain DNA that is not found in the plant prior to the genetic modification, or that is not found in the plant in the same form or in the same amount as prior to the genetic modification. For example, a gene encoding an enzyme from another source (e.g. from another organism, or from another plant species or variety) may be purified from the source and manipulated by well-known molecular biology techniques so as to be suitable for insertion into and expression in plant cells. For example, a gene from the novel AsA biosynthetic pathway of the present invention (such as the miox gene) may be isolated and purified from A. thaliana, cloned and manipulated using molecular biology techniques, inserted into a suitable vector, and then used to transform plant cells from another plant (e.g. tobacco, lettuce, etc.). Alternatively, the genetic modification may involve cloning of a gene from a plant species, genetic manipulation of the gene, and reinsertion and expression of the gene into the same species of plant. Thus, the transgenic plant may, prior to genetic manipulation, contain a gene encoding the same or a similar enzyme with which is it genetically transformed. However, the genetic modification contemplated by this invention confers some advantage that was not present in the native gene, such as causing the enzyme to be expressed in a manner that increases the level of AsA in the transgenic plant.

The gene or genes which are used to carry out genetic modification according to the present invention are those which encode enzymes of the novel AsA pathway. In one embodiment of the present invention, such genes are isolated from Arabidopsis thaliana. However, those of skill in the art will recognize that genes from the same pathway, but from different plant sources, may also be used in the practice of the present invention. The novel AsA biosynthetic pathway of the present invention is common to many (possibly all) species of plants and functional genes encoding useful enzymes from the pathway from any suitable source may be utilized. As a result, the precise sequence of a given gene may vary from plant to plant due to differences between species or varietal variation in sequence. Such natural variants of the genes in the biochemical pathway are intended to be encompassed by the present invention. In general, the genes will display homology to the genes first identified in A. thaliana, preferably from 50 to 100% homology, and more preferably 75 to 100% homology. Likewise, the polypeptide that is encoded by one of the genes of interest may vary in translated primary sequence from species to species, or among varieties. However, in general, they will display about 50 to 100% homology to the enzymes isolated from A. thaliana, and preferably about 75 to 100% homology. Further, genes encoding the MIOX enzyme from any other organism, for example, the pig (GenBank accession No. AF401311, Amer et al., 2001; reddy et al., 1981) may be utilized in the practice of the present invention.

The methodology for creating transgenic plants is well developed and well known to those of skill in the art. For example, dicotyledon plants such as soybean, squash, tobacco (Lin et al. 1995), and tomatoes can be transformed by Agrobacterium-mediated bacterial conjugation. (Miesfeld, 1999, and references therein). In this method, special laboratory strains of the soil bacterium Agrobacterium are used as a means to transfer DNA material directly from a recombinant bacterial plasmid into the host cell. DNA transferred by this method is stably integrated into the genome of the recipient plant cells, and plant regeneration in the presence of a selective marker (e.g. antibiotic resistance) produces transgenic plants.

Alternatively, for monocotyledon plants, such as rice (Lin and Assad-Garcia, 1996), corn, and wheat which may not be susceptible to Agrobacterium-mediated bacterial conjugation, genes may be inserted by such techniques as microinjection, electroporation or chemical transformation of plant cell protoplasts (Paredes-López, 1999 and references therein), or particle bombardment using biolistic devices (Miesfeld, 1999; Paredes-López, 1999; and references therein). Monocotyledon crop plants have now been increasingly transformed with Agrobacterium (Hiei, 1997) as well.

In order to insert a gene of the AsA biosynthetic pathway into a host plant, the gene may be identified, isolated and incorporated into a suitable construct such as a vector. Techniques for manipulating DNA sequences (e.g. restriction digests, ligation reactions, and the like) are well known and readily available to those of skill in the art. For example, Sambrook et al., 1989. Suitable vectors for use in the methods of the present invention are well known to those of skill in the art.

Further, such vector constructs may include various elements that are necessary or useful for the expression of the gene of interest. Examples of such elements include promoters, enhancer elements, terminators, targeting sequences, and the like. For example, a non-native (i.e. not associated with the gene in nature) constitutive or “strong” promoter sequence may be added in order to cause increased levels of expression of the gene. Similarly, an inducible promoter responsive, for example, to environmental conditions such as oxidative stress, may be inserted in order to make selective expression of the gene possible. Other types of genetic modifications that may be used to genetically modify the genes of interest of the present invention include but are not limited to the addition of developmentally regulated promoters to make possible selective expression at a particular time and/or location within the plant. All such potential genetic modifications are intended to be encompassed by the present invention, and any such useful elements may be incorporated into the constructs which house the gene of interest in the practice of the present invention. Further, those of skill in the art will recognize that a plant may be genetically modified to contain more than one gene of interest (i.e. several different genes of interest), and a single gene of interest may be present in more than one copy in the plant. Further, multiple copies of one gene of interest may be included in a single construct, or copies of more than one gene of interest may be included on a single construct. The gene(s) of interest may be retained in the host plant extrachromosomally, or may be integrated into the host plant genome.

In addition, those of skill in the art will recognize that many modifications of a gene sequence encoding an enzyme of interest (e.g., an enzyme of the novel AsA biosynthetic pathway) may be made that would still result in a gene/enzyme that would be suitable for use in the present invention. For example, alterations in the DNA sequence may be made for any of several reasons (for example, to produce a convenient restriction enzyme site) without affecting the amino acid sequence of the polypeptide translation product. Alternatively, changes may be made which alter the amino acid sequence of the polypeptide (either purposefully to change the polyepeptide sequence, or inadvertently due to a desired change in the DNA sequence) which still result in the production of a suitable, functional enzyme. For example, conservative amino acid substitutions may be made, or less conservative changes, such as the deletion or insertion of amino acids, may be carried out. For example, amino acids may be deleted from the amino or carboxy terminus of the polypeptide, or new sequences (e.g. targeting sequences) may be added to the polypeptide; or changes may be made to alter the stability of the mRNA or the protein. All such changes are intended to be encompassed by the present invention, so long as the resulting polypeptide is functionally expressed in the transgenic host plant and results in increased production of AsA in the transgenic host plant. In general, such changes will result in a polypeptide with about 85 to 100% homology to the naturally occurring enzyme, and preferably with about 95% homology. The amino acid homology of peptides can be readily determined by contrasting the amino acid sequences thereof by well-known techniques.

Further, by “a transgenic plant that has been genetically modified” we mean any part of the plant at any stage of the life cycle of the plant that has either been directly genetically manipulated, or the progeny of cells, plants, or parts of plants that have been so manipulated. The present invention thus encompasses transformed single cells, plants which are produced from transformed cells, (including all parts of the plant, e.g. leaves, roots, fruit, seeds, flowers, stalks, stems, cones, etc.), and any progeny of the transformed plants however produced (e.g. from seeds, by grafting, etc.), so long as the genetic modification is still retained within some part of the progeny.

The invention further provides a method of increasing the amount of AsA in a plant above endogenous levels. The method preferably includes genetically modifying the plant to contain at least one gene encoding an enzyme from the novel AsA biosynthetic pathway of the invention, the pathway being one which includes a myo-inositol oxygenase enzyme. Genetic modification of the plant in this manner results in production of AsA in the plant at a level higher than endogenous levels. By “endogenous level” we mean the amount of AsA that is produced by a corresponding plant (i.e., a plant of the same variety) that has not been genetically modified by insertion of such a gene. In other words, the level or amount of AsA produced in the genetically modified plant is greater than that produced in an equivalent plant that has not been genetically modified (engineered) in this manner. Those of skill in the art will recognize that the determination of whether or not the level or amount of AsA that is produced in a genetically modified plant is increased will typically be made by comparison to an otherwise identical (as nearly as possible) “control” (non-genetically modified) plant or group of plants. Such a control plant will be treated as nearly as possible in the same manner as the experimental, genetically modified plant or plants. The levels of AsA produced in each is assayed and compared, and will generally be considered as significantly increased if the level of AsA in the genetically modified plant is at least about 10% to about 100% or more higher than that of a corresponding non-genetically modified plant or plants, and preferably at least about 25% to about 100% or more higher. In a preferred embodiment, the genetically modified plant exhibits a 2 to 3-fold increase in AsA production. Further, AsA production need not be increased in all parts of the plant. Rather, the amount of AsA in one or more parts of the plant may be increased. For example, AsA production may be increased in the plant leaves, roots, flowers, fruit, seeds and the like, or in several or in all of these plant parts.

Examples of plants which may be genetically modified to produce increased levels of AsA include but are not limited to A. thaliana, tobacco (e.g. Nicotiana tabacum), lettuce (e.g. Lactuca sativa), as well as other diverse plants such as grains (e.g. rice, wheat, etc.), soybeans, fruit (e.g. apples, peaches, cherries, bananas, tomatoes, etc), vegetables (e.g. potatoes, carrots, corn, etc.), and decorative plants. In short, the present invention is applicable to any type of plant. The beneficial consequences of increasing the level of AsA in plants may include but are not limited to, for example, increasing the nutritional quality of the plant and increasing the overall health and/or tolerance to stress of the plant itself. For example, plants that are genetically engineered according to the method of the present invention may exhibit: increased shelf-life (in the case of both comestible produce and non-comestible plants such as decorative flowering plants; higher resistance to stresses during growth such as extremes of temperature, exposure to sun, and rainfall; shorter time to maturity in the field (e.g. shorter germination and/or maturation times); higher resistance to stresses after harvest, such as resistance to bruising or spoiling; etc.

The present invention also provides methods for reducing TSNAs in tobacco plants. This aspect of the invention involves genetically modifying tobacco plants to produce increased levels of AsA. By “reducing TSNAs in tobacco plants” we mean that the level of TSNAs in the leaves of the cured plants is about 5 to about 99% lower, and preferably about 20 to about 99% lower, and more preferably about 50 to about 99% lower, than the level in corresponding control tobacco plants that have not been genetically modified to produce increased levels of AsA. In the practice of the present invention, the tobacco plants can be transformed with any gene that results in an increase in the endogenous level of AsA in the plant, examples of which include but are not limited to the rodent L-gulono-gamma-lactone oxidase gene. In one embodiment, the level of AsA in tobacco plants is increased by transforming tobacco plants with at least one gene from the novel AsA biosynthetic pathway of the present invention. In one embodiment of the invention, the gene is that which encodes the enzyme myo-inositol oxygenase. In a preferred embodiment of this aspect of the invention, the tobacco plant is a variety of air-cured tobacco, examples of which include but are not limited to Burley variety VA501. However, those of skill in the art will recognize that the method need not be limited to air-cured varieties of tobacco.

Those of skill in the art will recognize that, in addition to genetically engineering plants to contain increased levels of AsA, it is also possible to select naturally occurring variants of plants that produce elevated levels of AsA, and to selectively breed these variants either with or without cross breeding them to each other. The present invention also encompasses such tobacco plants, particularly as they are used to decrease TSNAs. A skilled artisan will be well aware of techniques for selecting and propagating such tobacco plants. Tobacco plants that are identified in this manner will have elevated levels of AsA production in the range of about 10% to about 100% or more, and preferably at least about 25% to about 100% or more, compared to similar plants that are not selectively bred. As is the case for genetically engineered plants, any type of tobacco plant or tobacco variety may be selected in this manner, and the elevated AsA production may occur in any part of the plants, or in several or all parts of the plants, and their progeny.

### EXAMPLES

### Materials and Methods

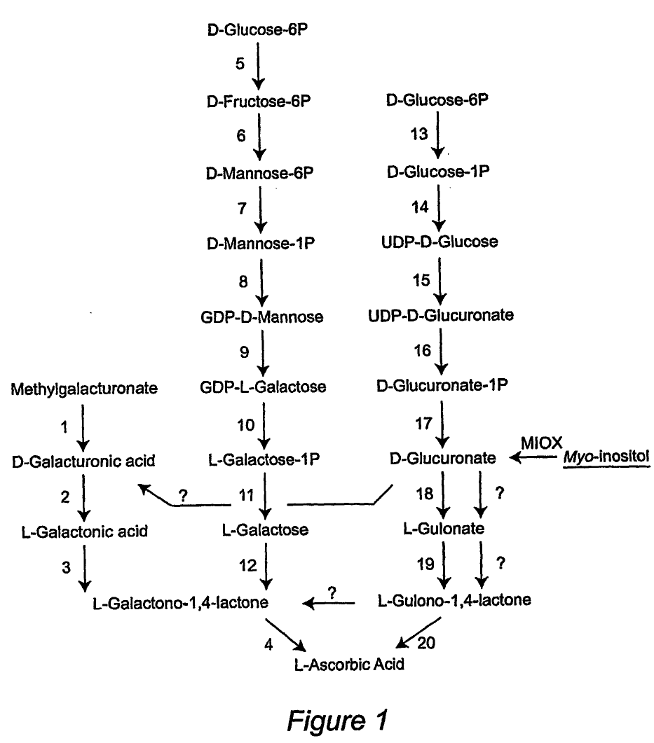

Background. The AsA biosynthetic pathways differ between animals and plants (FIG. 1). In animals, D-glucose is converted to AsA via D-glucuronic acid, L-gulonic acid, and L-guluno-1,4-lactone, which is then oxidized to AsA. In this pathway, the stereochemistry of the carbon skeleton of the primary substrate glucose is inverted in the final product. Feeding studies have shown that inversion of the glucose carbon skeleton does not occur during AsA biosynthesis in plants (Loewus 1963). Despite the importance of AsA in plant physiology and animal health, its biosynthetic pathway via GDP-mannose (GDP-Man) and L-galactose (L-Gal), was proposed only recently (Wheeler et al, 1998). According to this pathway GDP-Man is first converted to GDP-L-Gal by GDP-Man-3,5-epimerase. L-Gal is then formed from GDP-L-Gal by as yet uncharacterized steps. L-Gal is oxidized to L-galactono-1,4-lactone (L-GaIL) by L-galactose dehydrogenase and then to AsA by L-galactono-1,4-lactone dehydrogenase.

Although the AsA biosynthetic pathway proposed by Smirnoff-Wheeler (Wheeler et al, 1998) is consistent with most of the available data, there is growing evidence indicating the existence of other pathways operating in plants that contribute to the AsA pool. Tracer and feeding studies have shown conversion of methyl-D-galacturonate and D-glucuronolactone to AsA in detached leaves of different plant species (Loewus, 1963) and Arabidopsis cell cultures (Davey et al., 1999). Recently, the cloning of a D-galacturonic acid reductase from strawberry fruit, and its expression in A. thaliana, provided molecular evidence of the use of D-galacturonic acid as a precursor for AsA biosynthesis (Agius et al., 2003). A 4 to 7 fold increase in the AsA content was obtained in lettuce and tobacco plants after constitutive expression of the rat gene encoding L-guluno-1,4-lactone oxidase, the enzyme involved in the final step of the animal pathway (Jain and Nessler, 2000). It is still unclear if this enzyme works on the known plant precursor, L-GalL, or if plants can produce L-guluno-lactone. Additional evidence indicating the complex network of metabolic pathways leading to AsA comes from the analysis of the vitamin C-deficient A. thaliana mutants. The vtc2-1, vtc3-1, and vtc-4-1 mutants are defective in AsA biosynthesis, but when the activities of several of the proposed AsA biosynthetic enzymes have been measured, the results were not significantly different than those in wild type (Conklin et al., 2000; Smirnoff et al., 2001). None of these mutants appear to turn over AsA more rapidly than wild type.

The use of molecular tools to investigate the contribution of myo-inositol (MI) as a precursor of AsA biosynthesis in Arabidopsis is described in the Examples below.

### Example 1

### Isolation of A. thaliana MI Oxygenase (MIOX)

A full length cDNA that encodes a M oxygenase (MIOX, EC 1.13.99.1) from pig was recently isolated (GenBank accession no. AF401311), and characterized (Amer et al., 2001; Reddy et al, 1981). The Arabidopsis genome was searched (at TAIR (Huala et al. 2001) with the BLAST (Altschul et al., 1997) algorithm (TBLASTN version) for similar sequences. This resulted in five matching ORFs: At1g14520, At4g26260, At2g19800, At5g56640, and At5g08200, none of which have assigned functions. Alignment of these sequences with AF401311 revealed two segments for which the Arabidopsis sequences have very high similarity: 68-112 and 200-220. This high level of conservation suggests that these regions might be functional domains. These two conserved subsequences of AF401311 were used to interrogate InterPro (Apweiler et al., 2000), the integrated protein documentation resource. Subsequence 68-112 matched the ProDom (Corpet et al., 2000) domain PD037591, while 200-220 had no match. The protein family containing domain PD037591 includes five Arabidopsis sequences and one mRNA from Pinus radiata embryo (GenBank accession no. AF049069), which also has high similarity to the original pig MIOX gene. The domain is annotated as “kidney-specific”, presumably due to the origin of a number of animal sequences containing the domain (ProDom is annotated automatically). The five Arabidopsis sequences in this protein family correspond to four ORFs: At5g56640, At4g26260, At2g19800, At1g14520 (in two separate BAC sequences). At5g56640 and At4g26260 have the same domain structure, containing additional domains PD330223 and PD348512. At1g14520 also contains PD330223 and PD354868. At2g19800 contains only the common domain (PD037591), as does the P. radiata sequence.

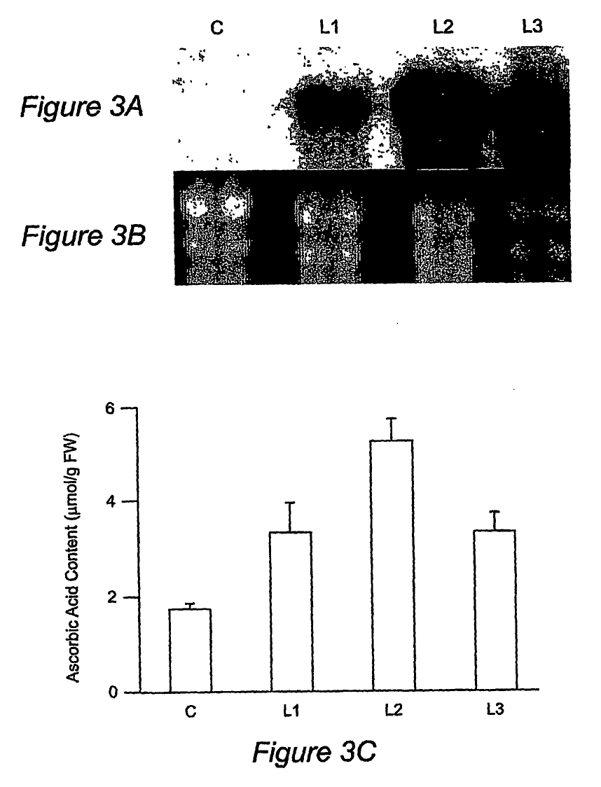

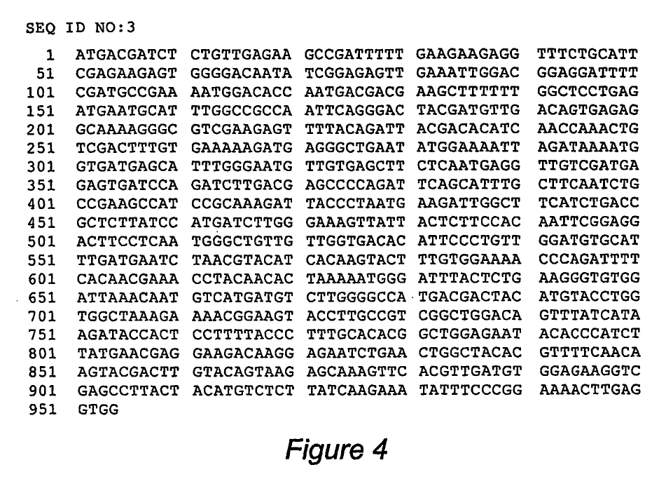

The coding region of the miox cDNA in chromosome 4 (miox4, GenBank accession no. At4g26260) of A. thaliana was isolated by PCR and sequenced. Specific primers for the putative miox gene in chromosome 4 (miox4) were designed with NcoI and BamHI sites added to the forward (MX4-5 CCCATGGCGATCTCTGTTGAG; SEQ ID NO:1) and reverse (MX4-3 CCGGATCCTCACCAC CTCAAG; SEQ ID NO:2) primers to facilitate sub-cloning. A 25 μl PCR reaction containing 3 μl of an A. thaliana mixed tissue cDNA library (CD4-7) from the Arabidopsis Biological Resource Center (ABRC, Columbus, Ohio) as template was performed with proofreading polymerase (Pfu Turbo DNA polymerase, Stratagene, La Jolla, Calif.). After denaturation at 94° C. for 5 min, amplification was performed by 30 cycles of 1 min at 94° C., 1 min at 50° C. and 2 min at 72° C., followed by 10 min at 72° C. The 957 bp PCR fragment was cloned into the pGEM-T Easy vector (Promega, Madison, Wis.), amplified in Escherichia coli DH5α and sequenced in both directions with T7 and SP6 primers using the ABI PRISM BigDye Terminator Cycle Sequencing Kit (E Applied Biosystems, Foster City, Calif.). A BLAST (Altschul et al., 1997) search with the 957 bp PCR product revealed three changes at bases 233, 759 and 901 when compared to the published sequence. Two of those changes caused a substitution at the amino acid level (Q₇₈ to R and K₃₀₀ to E, GenBank accession no. AY232552). The molecular mass based on the translated amino acid sequence for MIOX4 was calculated to be 37.061 Da with a theoretical pI of 4.83. The nucleic acid sequence (SEQ ID NO:3) and the amino acid sequence (SEQ ID NO:4) of the cDNA PCR product are given in FIGS. 3 and 4, respectively.

### Example 2

### Expression, Purification and Characterization of Recombinant A. thaliana MI-Oxygenase (MIOX)

MI oxygenase (MIOX) is an enzyme containing non-heme iron and catalyses a four-electron oxidation with the transfer of only one atom of oxygen into the product D-glucuronate. The identity of MIOX4 was confirmed by expressing the candidate ORF in E. coli. The NcoI/BamHI fragment corresponding to the coding region of the miox4 cDNA was sub-cloned into the pET32a(+) expression vector (Novagen, Madison, Wis.) placing it in frame with the 3′-end of the E. coli thioredoxin gene and a linker that includes a His-Tag sequence and the recognition sequence for protease enterokinase.

For expression, the pET32a:miox4 construct was transformed into E. coli BL21-Codon Plus(DE3) (Stratagene) heat-shock competent cells and a positive colony was grown overnight in LB/ampicillin (100 mg/l) medium at 37° C. and 250 rpm. This starter culture (10 mL) was used to inoculate 500 mL of LB/ampicillin medium supplemented with 250 mM sucrose, 250 mM NaCl, 2 mM glutathione and 1 mM proline and grown at 37° C. until OD₆₀₀ reached 0.5. Supplementing the LB medium with this cocktail of folding promoting agents increased the amount of soluble recombinant protein facilitating further purification steps. The culture was induced with 0.5 mM isopropyl β-D-thiogalactoside (IPTG) for 1 h and the cells were recovered by centrifugation at 10,000 g. Significant fusion protein expression was observed from the pET32a:miox4 construct in E. coli after 1 hour of induction with IPTG by SDS-PAGE analysis.

The pellet obtained was suspended and sonicated in 50 mM Tris-HCl pH 7.0, 500 mM NaCl, 10% glycerol, 10 mM β-mercaptoethanol, 5 mM imidazole, and 0.1 mg/ml lysozyme, and the resulting lysate was separated into soluble and insoluble fractions by centrifugation. The fusion protein was purified using BD-Talon cobalt-based affinity chromatography resin (BD Biosciences Clontech, Palo Alto, Calif.) according to the protocol supplied by the manufacturer.

Eluted fractions were collected, dialyzed against 50 mM Tris-HCl pH 7.2, 50 mM KCl, 1 mM glutathione, and MIOX4 was then cleaved away from the fusion protein by digestion with enterokinase (1 U/50 μg recombinant protein, Sigma, St. Louis, Mo.). The purity and molecular weight of the proteins in the column fractions were examined by SDS-PAGE (10% w/v). Protein concentration was determined using the Bradford assay and bovine serum albumin as a standard (Bradford, 1976).

MIOX activity was measured using an orcinol-based assay as described previously (Reddy et al, 1981). The MIOX 4 specific activity obtained using the orcinol-based assay (2174.28±219.65 mmol/min mg protein, n=3, is slightly higher compared to the activity of the only other MIOX expressed in bacteria, the enzyme from pig (1546 mmol/min mg protein, (Arner et al., 2001). The specific activity calculated for the MIOX4 recombinant protein is approximately 38 times higher compared to the activity reported for the native MIOX purified from oat seedlings (Koller et al., 1976). This may be due to differences in the methods of purification used, to differences in the properties of the enzymes (the molecular weight reported for the oat MIOX is 62 kDa), or to the poor stability of the oat enzyme preparation (Koller et al., 1976).

There is evidence indicating that the native MIOX enzyme from pigs is found in a complex with glucuronate reductase, the enzyme responsible for the second step of myo-inositol catabolism, and that this reductase prefers the acyclic form of glucuronate. From this, it is presumed that MIOX can transfer the acyclic form of the substrate directly to the reductase in the complex (Reddy et al., 1981). It is possible that MIOX4 also forms part of a metabolon in Arabidopsis.

### Example 3

### Expression Pattern of miox4 in Transformed Plants

Arabidopsis tissue samples were collected from plants grown in soil for 6 weeks under greenhouse conditions. Total RNA was extracted from above ground organs and roots by either TRI Reagent (Sigma) or RNeasy kit (Qiagen) following the instructions provided by the manufacturers, and from green siliques by a SDS/phenol method (Takahashi et al, 1991). Messenger RNA was isolated using the Micro-FastTract 2.0 mRNA Isolation Kit (Invitrogen, Carlsbad, Calif.), acording to the protocol supplied by the manufacturer. RNA was suspended in water and precipitated twice with 7 M ammonium acetate and 100% ethanol. RNA yield was quantified spectrophotometrically.

For Northern analysis, 2 μg of mRNA (tissues) or 8 μg of total RNA (over-expressers) of total RNA was separated on 1.2% (w/v) denaturing (formaldehyde) agarose gels, and transferred to nylon membranes (Hybond-N⁺, Amersham, Piscataway, N.J.). Membranes were pre-hybridized for 2 h at 65° C. and hybridized overnight at 65° C. in 500 mM sodium phosphate buffer pH 7.2 containing 7% SDS, 1% bovine serum albumin and 1 mM EDTA. The miox4 insert was excised from pGEM-T using NcoI/BamHI, purified after gel electrophoresis, and labeled with ³²P using Primer-It RmT Random Primer Labeling Kit (Stratagene). A β-ATPase PCR product was amplified as described previously (Riechers and Timko, 1999), purified after gel electrophoresis, labeled with ³²P, and used as a loading control in the experiments with mRNA. Following hybridization, filters were washed five times for 30 min at 65° C. in 20 mM sodium phosphate buffer pH 7.2, 0.1% SDS, 33 mM NaCl, and 1 mM EDTA and subjected to autoradiography (Kodak X-Omat AR film, Kodak, Rochester, N.Y.) between intensifying screens for 20 hours at −80° C.

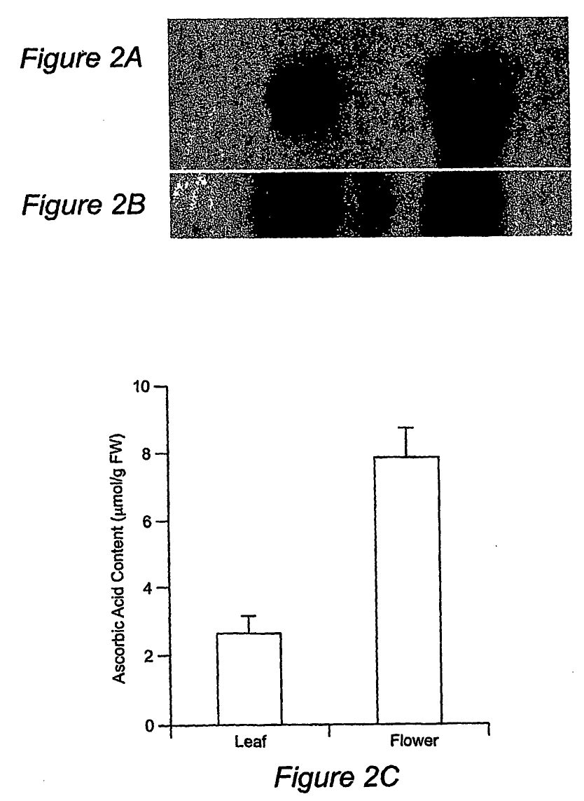

Hybridization experiments performed with mRNA purified from different tissues of Arabidopsis plants show that miox4 is predominantly expressed in flowers and leaves (FIG. 2). The observed expression pattern for miox4 correlates with the high demand for AsA of those plant tissues.

The AsA content was measured by the ascorbate oxidase assay (Rao and Ormrod, 1995). Plant extracts were made from tissue frozen in liquid nitrogen, and ground in 6 mM meta-phosphoric acid. Total ascorbic acid was determined by measuring the absorbance at 265 nm after addition of 1 U of ascorbate oxidase (Sigma) to the reaction medium containing the plant extract and 100 mM potassium phosphate, pH 5.6.

The AsA content of A. thaliana wild type flowers has been found here (FIG. 2, panel c) and by others (Conklin et al., 2000), to be 2 to 3 times greater than the AsA content of the leaves. In addition, a recent study reports transport of AsA from source leaf phloem to root tips, shoots and floral organs (Franceschi and Tarlyn, 2002). This is likely due to a greater demand for AsA in reproductive and actively growing tissues, which have higher metabolic rates, and because of that, a higher demand of antioxidants. Such tissues have increased rates of cell expansion and division and AsA is thought to have a role in these processes (Smirnoff et al., 2001; Arrigoni and De Tullio, 2002). Analysis of the AsA content of different tissues of the vtc2-1 and vtc2-2 Arabidopsis mutants shows a relatively higher level of AsA in flowers and siliques over that of mature leaves, despite their overall AsA deficiency, implying that these mutants retain an organ-specific control mechanism (Conklin et al., 2000). The still limited knowledge concerning the AsA route(s) of synthesis in plants has been generated mostly from studying leaf tissue. The possibility of alternative AsA biosynthetic pathways operating in specific organs or tissues requires further examination.

### Example 4

### Increased Production of AsA in Plants Transformed with miox4 ORF

The miox4 insert was cloned into the NcoI/BamHI sites of pRTL2 (Restrepo et al., 1990) placing it under the control of CaMV 35S promoter with duplicated enhancer between the 5′ tobacco etch virus (TEV) leader and the 3′ 35S polyadenylation signal. A PstI fragment including the promoter::miox4::terminator insert was sub-cloned into the binary vector pCAMBIA1300 and transformed into Agrobacterium tumefaciens strain GV3101. A. thaliana var. Columbia plants were transformed with pCAMBIA1300:miox4 construct via the floral dip method (Clough and Bent, 1998). Seedlings were selected on MS (Murashige and Skoog, 1962) plates containing 500 mg/l carbenecillin and 25 mg/l hygromycin. Both primary transformants and their progeny were used for RNA gel blot analysis and AsA assays.

To study the contribution of MI as a precursor of AsA biosynthesis, the miox4 ORF was expressed under the control of the strong constitutive 35S promoter in A. thaliana plants (see Materials and Methods). Analysis of three independent lines (L1, L2 and L3) of primary transformants and their progeny reveals a 2 to 3-fold increase in the AsA content of the leaves compared to wild type, and a line transformed with the empty vector pCAMBIA1380 grown under similar conditions (FIG. 3, panel c). This higher AsA content of the leaves of the over-expressors correlates with the amount of miox4 message detected by hybridization experiments (FIG. 3, panel a). Tracer and feeding studies previously performed with strawberry fruits and parsley leaves (Loewus, 1963) and Arabidopsis cell cultures (Davey et al, 1999) have failed to detect formation of AsA from M. This can be due to the relatively low amount of label used in experiments performed with leaves and fruits (Loewus, 1963) or to differences in the expression of the miox4 gene in undifferentiated cells growing in suspension (Davey et al, 1999).

Our results demonstrate the feasibility of using this gene to engineer increased vitamin C levels in plants. The evidence here presented also indicates that MI, a multifunctional molecule in plant biochemistry and physiology can be used as a precursor of ascorbic acid biosynthesis in Arabidopsis.

### Example 5

### Reduction of TSNAs in Leaves of Transformed Tobacco Plants

The tobacco variety, Nicotiana tabacum cultivar VA509 was transformed as previously described (Jain and Nessler, 2000) using disarmed A. tumefaciens with cDNA from brown rat Rattus norvegicus encoding L-gulono-gamma-lactone oxidase (GLOase), the terminal enzyme in the animal AsA biosynthetic pathway. The gene was housed in the pGLO173 construct. Expression of the gene was driven by the 35S 5′ promoter from cauliflower mosaic virus (CaMV) with the noncoding 5′ leader enhancer sequence from tobacco etch virus (TEV). The terminator sequence was 35S 3′ from CaMV. The selectable markers were as follows: promoter, nopaline synthase (nos) 5′ from A. tumefaciens T-DNA; gene, neomycin phosphotransferase (nptII) from E. coli Tn5; terminator, nopaline synthase (nos) 3′ from A. tumefaciens T-DNA. The transformed line was designated GLO-11.

Seedlings were germinated in the early spring and were transplanted to the field in May. The field plots consist of a single plant row of 20 plants for each genotype and a row of control plants for a total of 80 plants per location. Plants will be treated as normal, commercially grown Burley tobacco and not permitted to set seed. The normal Burley growing season finishes in late August to late September. Accordingly, harvesting is completed by approximately October 1^{st}. Plants are harvested, air-cured and the cured leaf is collected for chemical analysis. Chemical analyses include the determination of Vitamin C levels in the leaves and the level of TSNAs produced in the leaves prior to, during and after curing.

At all times of testing, the amount of AsA detected in the leaves of the transformed tobacco plants is significantly elevated (i.e. at least about 8-fold higher) compared to that of corresponding control (non-transformed) tobacco plants. Further, the level of TSNAs in the transformed tobacco plant leaves is significantly less (i.e. at least about 3-fold lower) than the level of TSNAs that is detected in the leaves of non-transformed tobacco plants, both during and at the end of the curing process.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Agius F. et al., Nat. Biotechnol. 21, 177 (2003).
Altschul S. F. et al., Nuc. Acids Res. 25, 3389 (1997).
Apweiler R. et al., Bioinformatics 16, 1145 (2000).
Arner R. J. et al., Biochem. J. 360, 313 (2001).
Arrigoni, O. and M. C. De Tullio, Biochim. Biophys. Acta 1569, 1 (2002).
Bradford, M. M. Anal. Biochem. 72, 248 (1976).
Clough, S. J. and A. F. Bent, Plant J. 16, 735 (1998).
Conklin P. L. et al., Genetics 154, 847 (2000).
Corpet F. et al., Nuc. Acids Res. 28, 267 (2000).
Davey M. W. et al., Plant Physiol. 121, 535 (1999).
Franceschi, J. K. and N. M. Tarlyn, Plant Physiol. 130, 649 (2002).
Hiei, Y et al. Plant Mol. Biol. 35, 205-218 (1997).
Huala E. et al., Nuc. Acids Res. 29, 102 (2001).
Jain, A. K. and C. L. Nessler, Mol. Breed. 6, 73 (2000).
Koller, E., F. Koller and O. Hoffman-Ostenhof, Mol. Cell. Biochem. 10, 33 (1976).
Lin, J.-J., M. Assad-Garcia, N. and J. Kuo. Plant Sci. 109, 171 (1995).
Lin, J.-J. and N. Assad-Garcia, In Vitro 32, 35A (1996).
Loewus, F. A. and M. W. Loewus, CRC Crit. Rev. Plant Sci. 5, 101 (1987).
Loewus, F. A. Phytochem. 2, 109 (1963).
Miesfeld, R. L. Applied Molecular Genetics, Wiley-Liss, publisher, pp. 205-235 1999.
Murashige, T., and F. A. Skoog, Physiol. Plant 15, 4763 (1962).
Paredes-López, ed. Molecular Biotechnology for Plant Food Production, Technomic Publishing, Inc. 1999.
Pastori G. M. et al., Plant Cell 15, 939 (2003).
Rao, M. and D. P. Ormrod, Photochem. Photobiol. 61, 71 (1995).
Reddy, C. C., J. S. Swan, and G. A. Hamilton, J. Biol. Chem. 256, 8510 (1981).
Restrepo, M. S. et al., Plant Cell 2, 987 (1990).
Riechers, D. E. and M. P. Timko, Plant Mol. Biol. 41, 387 (1999)
Rundlof, T. et al., J Agric Food Chem. 48, 4381 (2000).
Sambrook, J., E. F. Fritsch, and T. Maniatis, In Molecular Cloning: A Laboratory Manual, Second edition, Cold Spring Harbor Laboratory, Cold Spring Harbor, N.Y., 1989.
Smirnoff, N., P. L. Conklin, and F. A. Loewus, Annu. Rev. Plant Physiol. Plant Mol. Biol. 52, 437 (2001).
Smirnoff, N. and G. L. Wheeler, Crit. Rev. Biochem. Mol. Biol. 35, 291 (2000).
Smirnoff, N. Curr. Opin. Plant Biol. 3, 229 (2000).
Takahashi,m T. et al., Nature 393, 365 (1998).
Wheeler, G. L., M. A. Jones and N. Smirnoff, Nature 393, 365 (1998).

We claim:

1. A plant that is genetically modified to include at least one gene encoding an enzyme from a vitamin C biosynthetic pathway, wherein said pathway includes a myo-inositol oxygenase enzyme.

2. The plant of claim 1, wherein said plant includes more than one copy of said gene.

3. The plant of claim 1, wherein said plant further includes a means to enhance transcription of said gene or genes.

4. The plant of claim 1, wherein said plant is selected from the group consisting of lettuce, tobacco, and Arabidopsis.

5. The plant of claim 1, wherein said plant is a tobacco plant.

6. The plant of claim 1, wherein said at least one gene encodes a myo-inositol oxygenase enzyme.

7. A method of increasing an endogenous level of vitamin C in a plant, comprising the step of
genetically modifying said plant to contain at least one gene encoding an enzyme from a vitamin C biosynthetic pathway, wherein said pathway includes a myo-inositol oxygenase enzyme, and wherein said step of genetically modifying said plant results in increasing the intrinsic level of vitamin C in said plant.

8. The method of claim 7, wherein said plant contains more than one copy of said gene.

9. The method of claim 7, wherein said plant further includes a means to enhance transcription of said gene or genes.

10. The method of claim 7, wherein said plant is selected from the group consisting of lettuce, tobacco, and Arabidopsis.

11. The method of claim 7, wherein said plant is a tobacco plant.

12. The method of claim 7, wherein said at least one gene encodes a myo-inositol oxygenase enzyme.

13. A method for reducing TSNAs in air cured tobacco, comprising the step of
genetically engineering said tobacco to include at least one gene in a vitamin C biosynthetic pathway, wherein said step of genetically engineering said tobacco results in reduced levels of TSNAs in said tobacco.

14. The plant of claim 13, wherein said tobacco includes more than one copy of said gene.

15. The plant of claim 13, wherein said tobacco further includes a means to enhance transcription of said gene or genes.

16. The method of claim 13, wherein said pathway includes a myo-inositol oxygenase enzyme.

17. The method of claim 13, wherein said pathway includes a L-gulono-gamma-lactone oxidase enzyme.

18. The method of claim 13, wherein said at least one gene is rodent L-gulono-gamma-lactone oxidase enzyme.

19. The method of claim 13, wherein said at least one gene encodes a myo-inositol oxygenase enzyme.

20. The method of claim 13, wherein said step of genetically engineering said tobacco results in an increase in an endogenous level of vitamin C in said tobacco.

21. A tobacco plant that produces elevated levels of vitamin C.

22. The tobacco plant of claim 21 wherein said plant is produced by genetic engineering.

23. The tobacco plant of claim 21 wherein said plant is produced by selective breeding.
